(19) United States
(12) Patent Application Publication
(10) Pub. No.: US 2012/0321703 A1
(43) Pub. Date: Dec. 20, 2012

(54) CAPSULE FOR ORAL PET MEDICINE

(76) Inventor: Daniel KOURI, Blainville (CA)

(21) Appl. No.: 13/524,091

(22) Filed: Jun. 15, 2012

Related U.S. Application Data
(60) Provisional application No. 61/497,712, filed on Jun. 16, 2011.

(51) Int. Cl. A61K 9/48

(52) U.S. Cl. 424/451

(57) ABSTRACT

A capsule for the administration of pet medicine is described. The capsule comprises a main hollow body containing a liquid ingredient and having the configuration of a pet treat for receiving and concealing therein a pet medicine. The liquid ingredient contained in the capsule, with the medicine therein, is configured to be frozen and orally ingested by the pet. A method of orally administering medicine to a pet is also described.

### TECHNICAL FIELD

The present disclosure relates to pet medicine. More specifically, but not exclusively, the present disclosure relates to the oral administration of pet medicine. Still more specifically, yet not exclusively, the present disclosure relates to a capsule for orally administering medicine to pets such as dogs, for example.

### BACKGROUND

There exists a variety of pet medication for curing or preventing heartworms, fleas, ear mites, sarcoptic mange, certain types of ticks, hookworms, roundworms and a variety of other animal ailments.

Many drugs are applied topically yet oral administration remains far more effective regarding certain active ingredients in order for treating certain medical conditions.

A drawback with these oral administered medicaments is that it is difficult to have animals ingest them and thus, many pet owners turn to the less efficient topical methods.

### SUMMARY

In accordance with an aspect of the present disclosure there is provided a capsule for the administration of pet medicine. The capsule comprises a main hollow body shaped as a pet treat for receiving therein a liquid ingredient and the pet medicine. The liquid ingredient and the medicine are configured to be frozen and orally ingested by the pet.

In accordance with another aspect of the present disclosure, there is also provided a method for orally administering medicine to a pet. Medicine is inserted into a capsule having the shape of a pet treat, the capsule containing a liquid ingredient. The capsule is frozen. The pet is then introduced to the content of the capsule.

The foregoing and other features will become more apparent upon reading of the following non-restrictive description of illustrative embodiments thereof, given by way of example only with reference to the accompanying drawings.

### BRIEF DESCRIPTION OF THE FIGURES

Embodiments of the disclosure will be described by way of example only with reference to the accompanying drawings, in which:

FIG. 1 shows a capsule in accordance with a non-restrictive illustrative embodiment of the present disclosure;

FIG. 2 shows a variety of differently shaped capsules in accordance with other non-restrictive illustrative embodiments of the present disclosure;

FIG. 3 is a photograph showing a capsule mold and a void left by removal of another capsule mold;

FIG. 4 is a photograph showing a perspective view of a capsule mold; and

FIG. 5 is a photograph showing a perspective view of an array of capsule molds.

### DETAILED DESCRIPTION

The present disclosure provides a capsule for the oral administration of medicine by pets. The present disclosure also provides a method for the oral administration of medicine by pets.

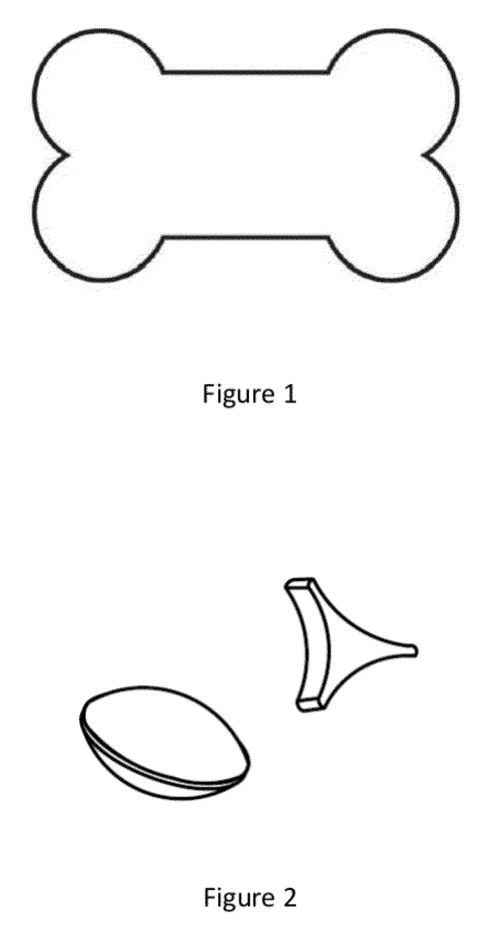

With respect to FIG. 1, there is shown a capsule in the form of a pet treat. In this example, the capsule is in the form of a dog bone which is typically fed to dogs. As shown in FIG. 2, the capsule may be given any other attractive shape. The capsule is made of plastic or similar material and is filled with liquid ingredients which provide a taste and scent that will attract the subject pet. A variety of ingredients well known in the art can be used to produce the present capsule.

The capsule comprises a shell-like structure with a hollow interior for containing the above mentioned liquid ingredients. In one embodiment, medication in liquid form may be inserted therein, for example using a syringe. The capsule may have a body having a wall whose thickness is thick enough so as to avoid liquid medicine that is inserted therein via the syringe to leak out. In another embodiment, medication may be pre-mixed with the above mentioned liquid ingredients at the time of manufacturing of the capsule. The skilled artisan will readily understand that a capsule including a hollow interior can be constructed by a variety of known ways.

The capsule along with the medicine can be frozen and provided as a treat to the pet.

In a further embodiment, the capsule can be provided in separate body portions that can be reconnected thereby allowing putting therein a powder or non-liquid medicament.

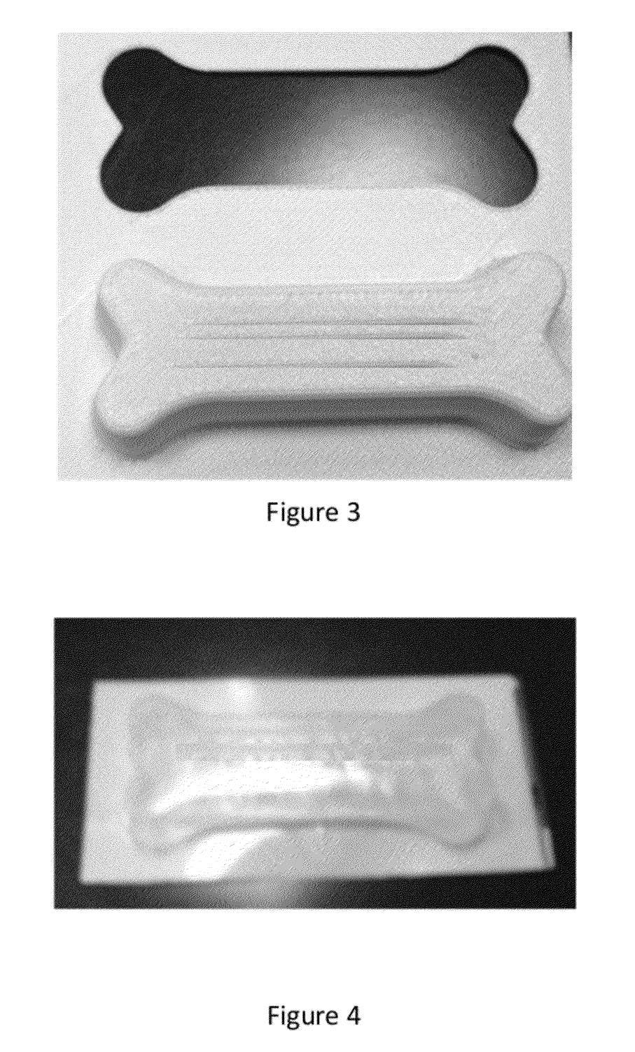

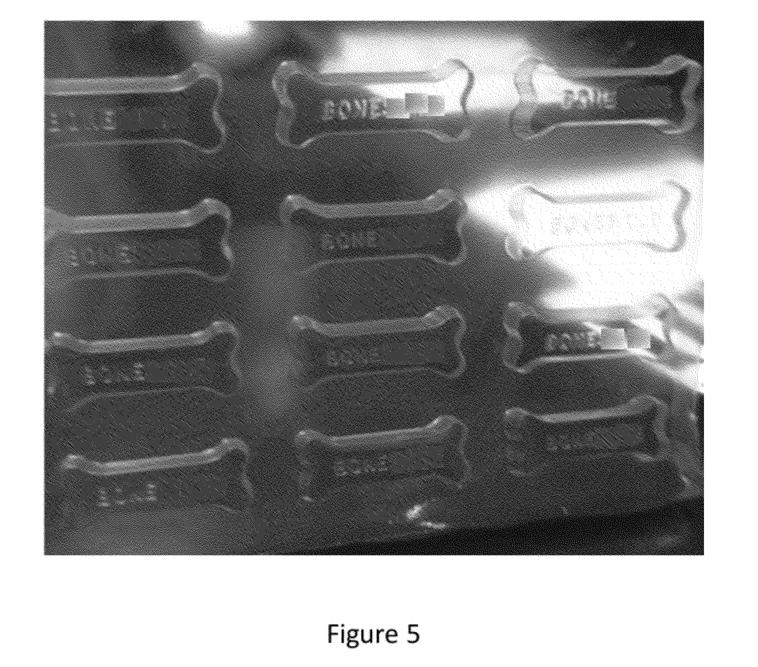

The following Figures provide added examples for the construction of the capsule. FIG. 3 is a photograph showing a capsule mold and a void left by removal of another capsule mold. FIG. 4 is a photograph showing a perspective view of a capsule mold. FIG. 5 is a photograph showing a perspective view of an array of capsule molds.

Those of ordinary skill in the art will appreciate that, in the foregoing description, the expression “liquid ingredients” may be understood as having various levels of fluidity and may be substituted with variants including syrupy ingredients, gummy ingredients, waxy ingredients, and the like. The capsule may be filled with ingredients that provide a taste and scent that will attract the subject pet, these ingredients having various levels of fluidity while being able to accommodate injection of pet medication before freezing.

There is also provided a method for orally administering medicine to pets. The method comprising inserting medicine into a capsule having the shape of a pet treat and containing a liquid ingredient that appeals to a pet, freezing the capsule, and introducing the pet to the capsule containing the medicine. The method may comprise a step of injecting the medicine into the capsule before freezing. Alternatively, the medicine may be premixed with the liquid ingredient.

Although the present disclosure has been described hereinabove by way of non-restrictive, illustrative embodiments thereof, these embodiments may be modified at will within the scope of the appended claims without departing from the spirit and nature of the present disclosure.

What is claimed is:

1. A capsule for the administration of pet medicine, comprising:
a main hollow body shaped as a pet treat for receiving therein a liquid ingredient and the pet medicine;
wherein the liquid ingredient and the medicine are configured to be frozen and orally ingested by the pet.

2. The capsule of claims 1, wherein a wall defining a body the capsule body is configured so as to allow the pet medicine in liquid form to be inserted in the capsule via a syringe.

3. The capsule of claim 2, wherein a thickness of the wall defining the body of the capsule is thick enough so as to avoid leakage of the pet medicine.

4. The capsule of claim 1, wherein the body of the capsule is provided in separate body portions that can be reconnected thereby allowing to put therein the pet medicine in powder or non-liquid form.

5. A method for orally administering medicine to a pet, comprising:
inserting medicine into a capsule having the shape of a pet treat, the capsule containing a liquid ingredient;
freezing the capsule; and
introducing the pet to the content of the capsule.
